(19) United States
(12) Patent Application Publication
(10) Pub. No.: US 2010/0211105 A1
(43) Pub. Date: Aug. 19, 2010

(54) Telescopic Rod For Posterior Dynamic Stabilization

(76) Inventors: Missoum Moumene, Newton, MA (US); Payman Afshari, South Easton, MA (US); Jonathan Fanger, Raynham, MA (US)

Correspondence Address: PHILIP S. JOHNSON;JOHNSON & JOHNSON, ONE JOHNSON & JOHNSON PLAZA, NEW BRUNSWICK, NJ, 08933-7003, US

(21) Appl. No.: 12/414,500

(22) Filed: Mar. 30, 2009

Related U.S. Application Data
(60) Provisional application No. 61/152,615, filed on Feb. 13, 2009.

(51) Int. Cl. A61B 17/70; A61B 17/88

(52) U.S. Cl. 606/258; 606/279

(57) ABSTRACT

A Posterior Dynamic Stabilization (PDS) device having a telescopic sub-assembly that allows axial movement but restricts bending, shear and in some configurations torsion, and an outer polymeric sleeve component that primarily resists elongation and axial rotation but also encapsulates the telescopic assembly to prevent tissue ingrowth.

### CONTINUING DATA

This divisional patent application claims priority from co-pending provisional U.S. Ser. No. 61/152,615, filed Feb. 13, 2009, entitled “Telescopic Rod for Posterior Dynamic Stabilization” (Moumene).

### BACKGROUND OF THE INVENTION

The vertebrae in a patient's spinal column are linked to one another by the disc and the facet joints, which control movement of the vertebrae relative to one another. Each vertebra has a pair of articulating surfaces located on the left side, and a pair of articulating surfaces located on the right side, and each pair includes a superior articular surface, which faces upward, and an inferior articular surface, which faces downward. Together the superior and inferior articular surfaces of adjacent vertebra form a facet joint. Facet joints are synovial joints, which means that each joint is surrounded by a capsule of connective tissue and produces a fluid to nourish and lubricate the joint. The joint surfaces are coated with cartilage allowing the joints to move or articulate relative to one another.

Diseased, degenerated, impaired, or otherwise painful facet joints and/or discs can require surgery to restore function to the three joint complex. Damaged, diseased levels in the spine were traditionally fused to one another. While such a technique may relieve pain, it effectively prevents motion between at least two vertebrae. As a result, additional stress may be applied to the adjoining levels, thereby potentially leading to further damage.

More recently, techniques have been developed to restore normal function to the facet joints. One such technique involves covering the facet joint with a cap to preserve the bony and articular structure. Capping techniques, however, are limited in use as they will not remove the source of the pain in osteoarthritic joints. Caps are also disadvantageous as they must be available in a variety of sizes and shapes to accommodate the wide variability in the anatomical morphology of the facets. Caps also have a tendency to loosen over time, potentially resulting in additional damage to the joint and/or the bone support structure containing the cap.

Other techniques for restoring the normal function to the posterior element involve arch replacement, in which superior and inferior prosthetic arches are implanted to extend across the vertebra typically between the spinous process. The arches can articulate relative to one another to replace the articulating function of the facet joints. One drawback of current articulating facet replacement devices, however, is that they require the facet joints to be resected. Moreover, alignment of the articulating surfaces with one another can be challenging.

Accordingly, there remains a need for improved systems and methods that are adapted to mimic the natural function of the facet joints.

Traditional spine fusion may result in iatrogenic instability at adjacent spine levels and subsequently require additional surgery to fuse more levels. Stabilization using more dynamic rods with traditional pedicle screw instrumentation may improve surgical outcomes and reduce additional surgeries for adjacent level degeneration.

U.S. Pat. No. 5,540,688 (Navas) discloses an intervertebral stabilization device, made in the form of a damper adapted to resist elastically, on the one hand, an elongation and, on the other hand, an axial compression without buckling, as well as of at least two implants anchored on two adjacent vertebrae.

U.S. Pat. No. 5,672,175 (Martin) discloses. a dynamic implanted spinal orthosis which preserves at least in part the natural physiological mobility of the vertebrae while effecting and maintaining a correction of the relative positions of the vertebrae without osteosynthesis, graft or fusion, comprising anchoring components fixed to the vertebrae and holding means associated with the anchoring components for holding the vertebrae with respect to each other in the corrected position, the holding means comprise an elastic return device for exerting elastic return forces, the orientation and magnitude of which are determined for holding the vertebrae in the corrected position against natural deforming forces for reducing the forces exerted on the vertebrae while preserving their mobility; also a procedure for maintaining a correction of the positions of the vertebrae for treating a deformation of the spine.

U.S. Pat. No. 5,375,823 (Navas) discloses an improved damper, of the type comprising elements for progressively resisting, in exponential manner, the advance of a piston under the effect of a force of axial compression, which functions as a stop opposing any displacement of the piston beyond a predetermined value, in an intervertebral stabilization device.

U.S. Pat. No. 5,562,737 (Graf) discloses An extra-discal intervertebral prosthesis comprising at least a partially closed, elongated body including a compression chamber having an elastic block at one end. The block has a free face abutted by a ball joint associated with a first of two fixation means engagable in spaced vertebrae of a patient.

U.S. Pat. No. 6,241,730 (Alby) discloses an intervertebral link device including at least one damper element constituted by a cage and a pin designed to be connected to bone anchor elements. The pin being engaged in a housing of the cage and being fitted with two elastically deformable members operating in opposition to an applied traction force or compression force. The damper element includes a pin that is mounted inside the cage by a joint allowing multidirectional relative pivoting between the pin and the cage, at least about the axes contained in a plane perpendicular to the pin and angular abutment between the cage and the pin enabling the multidirectional relative pivoting to be limited in amplitude to a determined value of about 4°

US Patent Publication No. 2003/0220643 (Ferree) discloses an apparatus for inhibiting full extension between upper and lower vertebral bodies, thereby preventing pain and other complications associated with spinal movement. In the preferred embodiment, the invention provides a generally transverse member extending between the spinous processes and lamina of the upper and lower vertebral bodies, thereby inhibiting full extension. Various embodiments of the invention may limit spinal flexion, rotation and/or lateral bending while preventing spinal extension. In the preferred embodiment, the transverse member is fixed between two opposing points on the lower vertebral body using pedicle screws, and a cushioning sleeve is used as a protective cover. The transverse member may be a rod or cable, and the apparatus may be used with a partial or full artificial disc replacement. To control spinal flexion, rotation and/or lateral bending one or more links may be fastened to an adjacent vertebral body, also preferably using a pedicle screw. Preferably a pair of opposing links are used between the upper and lower vertebral bodies for such purposes. Alternative embodiments use stretchable elements with or without a transverse member.

US Patent Publication No. 2004/0049189 (Le Couedic) discloses a connecting member for maintaining the spacing between at least two anchor members screwed into vertebrae. It comprises two rigid rod-forming parts made of a first material and each having a fixing, first portion adapted to be fixed into an anchor member and a fastening, second portion, said rods being aligned with each other and said fastening portions facing each other, and a connecting body made of a second material which is more elastically deformable than said first material and which interconnects said rigid parts by means of the facing fastening portions so that said connecting body is able to deform elastically, whereby the vertebrae, which are held spaced from each other, are movable relative to each other.

US Patent Publication No. 2005/0203519 (Harms) discloses a rod-shaped element for use in spinal or trauma surgery, having a first section for connecting to a first bone anchoring element and a second section for connecting to a second bone anchoring element. The rod-shaped element also includes a first elastic flexible element that is capable of elastic deformation when a force acts on it transverse to the rod axis. The first section and the second section are capable of shifting relative to each other in the direction of the rod axis. In a stabilization device for use in spinal or trauma surgery, the rod-shaped element allows for a controlled motion of the parts to be stabilized relative to each other so flexural motion is adjusted separately from the adjustment of the mobility in axial direction

US Patent Publication No. 2005/0288670 (Panjabi) discloses spine stabilization devices, systems and methods in which a single resilient member or spring is disposed on an elongate element that spans two attachment members attached to different spinal vertebrae. The elongate element passes through at least one of the two attachment members, permitting relative motion therebetween, and terminates in a stop or abutment. A second resilient member is disposed on the elongate element on an opposite side of the sliding attachment member, e.g., in an overhanging orientation. The two resilient members are capable of applying mutually opposing urging forces, and a compressive preload can be applied to one or both of the resilient members.

US Patent Publication No. 2006/0265074 (Krishna) discloses a lumbar disc prosthesis including a pair of disc members. The first member of the disc pair has a vertebral disc contact surface and a recessed portion on an opposing surface thereof. The second member of the disc pair has a vertebral disc contact surface and a protruding portion on an opposing surface thereof. The protruding portion of the second member engages with the recessed portion of the first member in use. Each of the first and second disc members are provided with at least three sections; a middle section and two end sections. The recessed and protruding portions are provided in the middle section of the respective disc members and each of the two end sections have a narrowing taper towards the ends of the disc members. The facet joint prosthesis includes a first member for attachment to a first posterior lumbar disc in use and a second member for attachment to a second posterior lumbar disc in use. At least a part of the first member is telescopically mounted in at least a part of the second member in use.

U.S. Pat. No. 7,476,238 (Panjabi) discloses a telescopic device with dual spring inside the assembly that controls the elongation and compression of the device.

U.S. Pat. No. 7,326,210 (Jahng) discloses a flexible rod system comprising of two compressible spacer one at each end. Jahng mentions two rigid ends and a flexible component disposed between the rigid ends.

U.S. Pat. No. 5,282,863 (Burton) discloses a non metallic flexible rod for vertebra stabilization.

US Published Patent Application No. 2006/0036240 (Colleran) discloses a dynamic device containing two rods that translate with one another.

US Published Patent Application No 2005/0171543 (Timm) discloses two elongating members that translate and a dynamic member as part of at least one elongate member. The dynamic member is shown as a spring in most images.

European Patent EP 0 669 109 B1 (Dubois Gilles) discloses a polymeric cylinder with a through strap that is anchored to pedicle screws.

### SUMMARY OF THE INVENTION

The present invention relates to the use of an external elastomeric sleeve surrounding a telescopic assembly within a dynamic stabilization (PDS) system.

The present invention provides a Posterior Dynamic Stabilization (PDS) Device that allows compression and elongation, which is a critical requirement for a PDS device as it allows pedicles to travel naturally in flexion and extension of the spine. This interpedicular travel preserves a more natural center of rotation unlike most other PDS devices that simply allow bending. The invention incorporates two main components: First, a telescopic sub-assembly that allows axial movement but restricts bending, shear and in some configurations torsion. Second, a polymer component that controls elongation but also encapsulates the telescopic assembly to prevent tissue ingrowth.

The primary embodiment of the invention is a single level rod used for PDS that contains the following components:

This invention comprises dynamic stabilizing rods that can be connected to pedicle screws and fixed to the spine. In a preferred embodiment, the rod comprises a hollow, closed-end cylinder component that is configured for attachment to a first pedicle screw, a solid rod component that is configured on a first end to slide freely within the hollow cylinder component, and configured at a second end for attachment to a second pedicle screw and an elastomer component contained encapsulating the rod from the closed end of the hollow cylinder and the second end of the solid rod component.

Preferably, the telescopic rod assembly of the present invention is composed of:
a) a metallic or polymer-based rod components that provide i) an inner rod with circular cross-section that serves as the core of the assembly, and ii) an outer tube that is bored out at the center to provide a sliding interface for the inner rod,
b) an elastomeric component that serves as a jacket/sleeve to encapsulates the inner and outer components; provides axial elongation stiffness for the assembly; and prevents tissue ingrowth;
c) an axial clearance space between the inner and outer rods that serves to limit axial compression, and,
d) a groove feature provides mechanical means to fasten the outer elastomer jacket to the inner and outer rods. If the set screw clamps the rod in this area it will further secure the elastomer jacket.

Therefore, in accordance with the present invention, there is provided a dynamic stabilization device comprising:
a) a first hollow cylinder having an open end, an intermediate annular portion and a closed end, the closed end defining an inner surface, the intermediate annular portion defining an outer annular surface and a first inner annular surface,
b) an inner rod having an outer diameter, a first end having a first end surface, and a second end, the first end of the inner rod being slidably received within the inner annular surface of the first hollow cylinder, and
c) a first elastomeric sleeve having a first end and a second end,
   wherein the first end of the first elastomeric sleeve is attached to the outer surface of the closed surface of the first hollow cylinder, and
   wherein the second end of the first elastomeric sleeve is attached to the outer surface of the second end of the inner rod.

### DESCRIPTION OF THE FIGURES

FIG. 1a discloses a longitudinal cross section of a device of the present invention during flexion.

FIG. 1b discloses a longitudinal cross section of a device of the present invention during extension.

FIG. 1c discloses a longitudinal cross section of an unloaded device of the present invention.

FIG. 2 discloses a side view of a device of the present invention having a pin and groove.

FIG. 3 discloses a perspective view of a device of the present invention attached to two bone anchors.

### DETAILED DESCRIPTION OF THE INVENTION

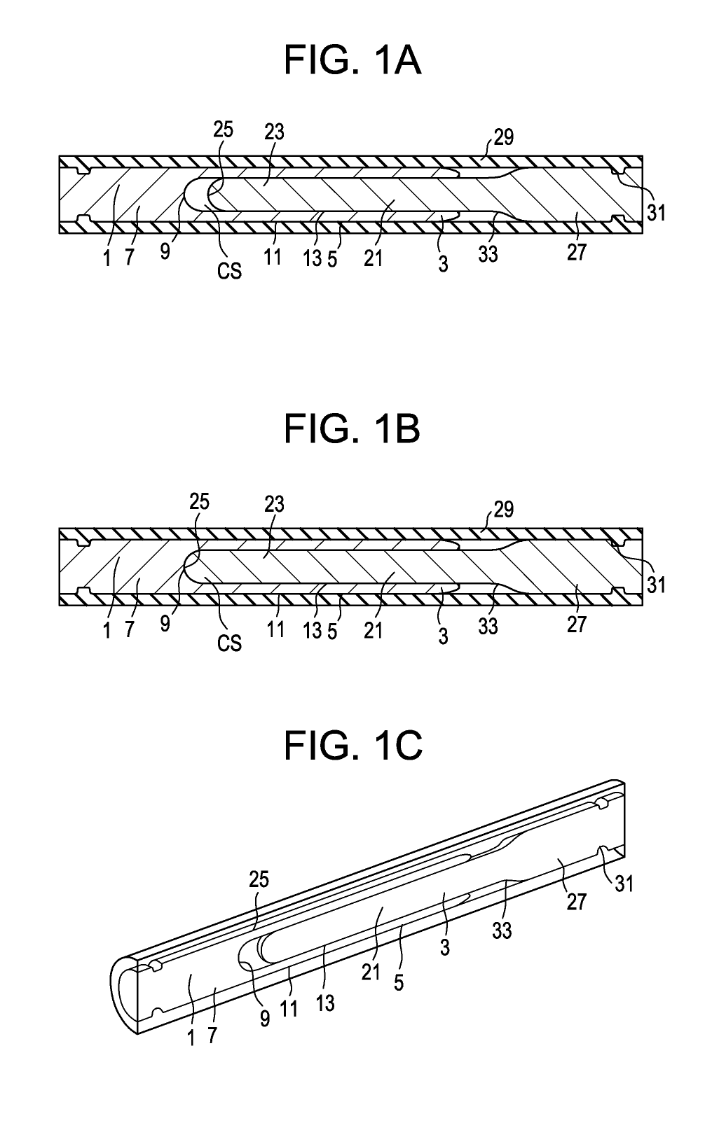

Now referring to FIGS. 1a-1c, there is provided a dynamic stabilization device comprising:
a) a first hollow cylinder 1 having an open end 3, an intermediate annular portion 5 and a closed end 7, the closed end defining an inner surface 9, the intermediate annular portion defining an outer annular surface 11 and a first inner annular surface 13,
b) an inner rod 21 having an outer diameter OD, a first end 23 having a first end surface 25, and a second end 27, the first end of the inner rod being slidably received within the inner annular surface of the first hollow cylinder, and
c) a first elastomeric sleeve 29 having a first end and a second end,
   wherein the first end of the first elastomeric sleeve is attached to the outer surface of the closed surface of the first hollow cylinder, and
   wherein the second end of the first elastomeric sleeve is attached to the outer surface of the second end of the inner rod.

FIGS. 1a-1c also disclose the presence (in FIG. 1a) and absence (in FIG. 1b) of a clearance space CS between the inner rod and outer hollow cylinder that serves to limit axial compression of the device. The clearance space is present when the spine is in flexion, and will approach a closed configuration as the rod bottoms out when the spine is in extension.

FIGS. 1a-1c also disclose a groove feature 31 that provides mechanical means to fasten the outer elastomer jacket to each of the inner rod and outer hollow cylinder. Alternatively, this feature could be a ridge or other similar feature instead of a groove. If a set screw (not shown) clamps the rod in this area, it will further secure the elastomer jacket.

FIGS. 1a-1c also disclose a bullet nose on the first end surface 25 of the inner rod. Likewise, the inner surface 9 of the hollow cylinder has a corresponding cup shape. The bullet nose and corresponding cup are advantageous because their correspondence reduces wear of these surfaces when they contact during spinal extension.

FIG. 1a-1c also disclose a transition radius in the intermediate section 33 of the inner rod to provide a better stress distribution as the rod moves along axis longitudinal axis from a smaller diameter first end to a larger diameter second end.

Preferably, the telescoping inner rod and outer hollow cylinder assembly may be constructed of non-circular cross-section to increase resistance to axial rotation. More preferably, the non-circular cross section is selected from the group consisting of an ellipse, a square, a polygon, and a T-slotted coupling.

Preferably, the telescoping components may be curved to better match the lordotic anatomy of the spine. Alternatively, the ends of these components may be canted.

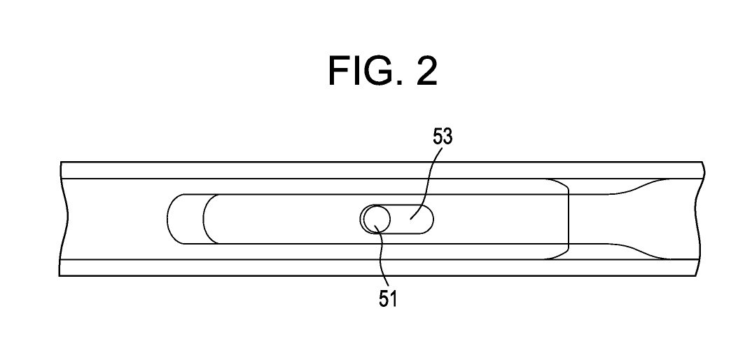

Preferably, and now referring to FIG. 2, the inner rod may have a pin 51 extending from its outer surface, and the outer hollow cylinder may have a corresponding groove 53 therein to limit the elongation of the telescoping assembly.

Preferably, the inner rod and outer hollow cylinder may have slotted features at their non-mating ends to allow the rod to mate with bone anchors that contain posts rather than slots. These slotted features are preferably used with bone anchors that are bolts (not polyaxial screws).

In some embodiments, an elastomeric bumper component may be present at the inner surface of the outer hollow cylinder, such that the elastomeric bumper would provide a cushioned stop when the inner rod reaches the bottom of the outer tube.

In some embodiments, an elastomeric ring may be assembled to the outside of the inner rod at its first end. This ring is another option for providing cushioning during full spine extension.

In some embodiments, an elastomeric ring may be provided around the necked region of the inner rod.

Various geometric features could also be machined into the outer surfaces of the inner rod and outer tube to mechanically connect the elastomer sleeve. For example, this feature could be a groove or ridge. In some embodiment, the feature is a boss provided to be perpendicular to the axis of the rod. This boss could serve two purposes. First, it could provide mechanical connection between the elastomeric component and telescoping assembly. Second, it could serve as a surface for tightening a set screw.

Various other features may be built into the outer or inner rod to limit elongation and to limit the amount of strain placed on the elastomer sleeve. These features may also contribute to axial rotation resistance. Such features may include a pin and slot, or a T-shape.

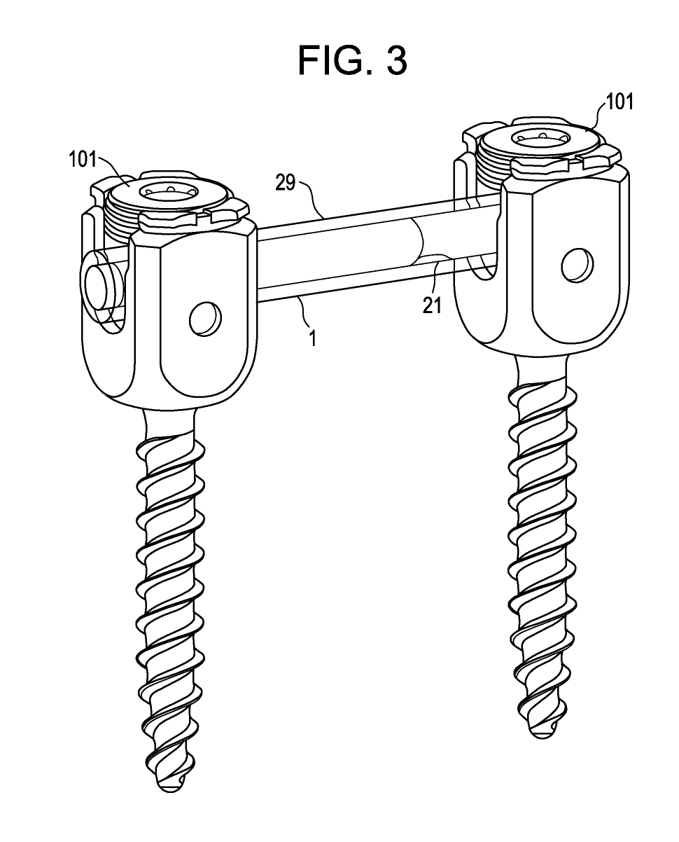

In some embodiments, a set screw may be provided in the bone anchor (see FIG. 3). The purpose of the set screw is to prevent rotational contact of the rod or hollow cylinder with the polymer jacket, which could generate torque that may damage the polymer jacket surface. The set screw may contain a washer or rotating saddle shape feature at its assembly-contacting surface that would allow axial load to be applied to the elastomeric sleeve without transferring torque. In some embodiments, a set screw may be provided that contains a small boss at its center, which will penetrate the elastomeric sleeve and minimize torque to the surface. In preferred embodiments, a pin feature could be paired with an inner rod/outer hollow cylinder assembly made from a polymer so that the pin could penetrate and bite the rod.

In general, the elastomer sleeve or jacket of the present invention may be constructed of polymers with various durometers. In some embodiments, a kit may be provided, with the sleeve being manufactured in a plurality of different durometers to provide varying stiffness for different patients and/or indications. In some embodiments, the thickness of the elastomer sleeve may be altered to achieve various stiffnesses. In some embodiments, a kit may be provided, with various sleeve to provide varying stiffnesses for different patients and/or indications. In preferred embodiments, the elastomer sleeve will run the full length of the rod/hollow cylinder assembly. However, it is also possible to design the sleeve so that it may only run part of the lengths of the inner rod and outer cylinder assembly.

In some embodiments, a deformable thin metallic sleeve is provided around the elastomeric sleeve so that compressive forces are not directed directly against the elastomeric sleeve.

Optionally, a hole is designed into the device to allow telescopic air movement to escape from the device.

In some embodiments, the outer hollow cylinder or inner rod may have an attachment feature for attachment to minimally invasive surgery (MIS) instruments that would allow passage of the assembly through an MIS portal. Assembly passage can at times be difficult and involve contact with metallic instruments, particularly screw extensions, that could damage the outer surface of the rod. Therefore, in some embodiments, an annular mating instrument may be provided that provides a protective shield for the polymeric jacket during assembly passage through the MIS portal.

In a multiple spinal level construct is desired, a double ended inner rod may be provided for a two-level assembly with opposing outer tube components articulating with each end of the rod. Similarly, three or more level assemblies may be constructed of a long inner rod with a stepped end. Outer tubes may be assembled and pinned in place for each level. An additional piece must be added to the opposite end to increase the diameter for bone anchor attachment.

Top-off rods may be constructed by providing an extended rod length from one side of the inner or outer rod. This rod length may or may not contain the elastomeric component.

Therefore, in accordance with the present invention, there is provided a dynamic stabilization device comprising:
a) a first hollow cylinder having an open end, an intermediate annular portion and a closed end, the closed end defining an inner surface, the intermediate annular portion defining an outer annular surface and a first inner annular surface,
b) a first rod having an outer diameter, a first end having a first end surface, and a second end, the first end of the first rod being slidably received within the inner annular surface of the first hollow cylinder, and
c) a second hollow cylinder having an open end, an intermediate annular portion and a closed end, the closed end defining an inner surface, the intermediate annular portion defining an outer annular surface and a second inner annular surface,
d) a first elastomeric sleeve having a first end and a second end,
   wherein the first end of the first elastomeric sleeve is attached to the closed end of the first hollow cylinder, and
   wherein the second end of the first elastomeric sleeve is attached to the closed end of the first hollow cylinder.

Also in accordance with the present invention, there is provided a dynamic stabilization device comprising:

a) a first rod having an outer diameter, an inner end having an inner end surface, and an outer end, the inner end of the first rod being slidably received within the first inner annular surface of the dual hollow cylinder, and
b) a second rod having an outer diameter, an inner end having an inner end surface, and an outer end, the inner end of the second rod being slidably received within the second inner annular surface of the dual hollow cylinder, and
c) a dual hollow cylinder having an first and second open ends defining first and second annular portions, an intermediate solid portion, the first and second annular portions defining first and second outer annular surfaces and first and second inner annular surfaces,
d) a first elastomeric sleeve having a first end and a second end,
wherein the first end of the first elastomeric sleeve is attached to the outer end of the first rod, and
wherein the second end of the first elastomeric sleeve is attached to the outer end of the second rod.
Also in accordance with the present invention, there is provided a dynamic stabilization device comprising:
a) a first hollow cylinder having an open end, an intermediate annular portion and a closed end, the closed end defining an inner surface, the intermediate annular portion defining an outer annular surface and a first inner annular surface,
b) a first rod having an outer diameter, a first end having a first end surface, and a second end having a second end surface.
c) a third component with a first rod end, and second hollow cylinder with an open end defining an inner surface, the annular portion defining an outer annular surface and an inner annular surface, the first rod end containing a first end forming a first outer diameter to be slidably received within the inner annular surface of the first hollow cylinder and the second hollow end slidably mated to the first rod.
d) a first elastomeric sleeve having a first end and a second end, wherein the first end of the first elastomeric sleeve is attached to the closed end of the first hollow cylinder and the second end of the first elastomeric sleeve attached to the second end surface of the first rod.

In one of the preferred embodiments of the present invention, the device possesses a number of advantageous features: It has an inner rod and an outer hollow cylinder that telescope with each other and are surrounded by an elastomeric jacket (sleeve). The axial stiffness of the assembly is controlled by the stiffness of the polymeric sleeve. In embodiments using a circular metallic rod and hollow cylinder, the torsion is also carried by the polymeric sleeve. In embodiments in which the rod has a circular cross section, the shear and bending stiffness of the assembly (tosional load of the spine) is primarily controlled by the rod and hollow cylinder forming the telescopic component. In embodiments in which the rod has a non-circular cross-section, the metallic rod and hollow cylinder will carry a slightly greater portion of the spine's torsional loads (and the polymeric jacket will slightly contribute to the assembly torsional stiffness). A clearance space between the ends of the metallic rods will act to limit the amount of compression in the assembly. Finally, a set screw located on the bone anchor can provide an clamping force perpendicular to the rod, but will not transfer torque to the elastomeric jacket surface.

One skilled in the art will appreciate that the device assembly may be configured for use with any type of bone anchor, e.g., bone screw or hook; mono-axial or polyaxial. Typically, a bone anchor assembly includes a bone screw, such as a pedicle screw, having a proximal head and a distal bone-engaging portion, which may be an externally threaded screw shank. The bone screw assembly may also have a receiving member that is configured to receive and couple a spinal fixation element, such as a spinal rod or spinal plate, to the bone anchor assembly.

The receiving member may be coupled to the bone anchor in any well-known conventional manner. For example, the bone anchor assembly may be poly-axial, as in the present exemplary embodiment in which the bone anchor may be adjustable to multiple angles relative to the receiving member, or the bone anchor assembly may be mono-axial, e.g., the bone anchor is fixed relative to the receiving member. An exemplary poly-axial bone screw is described U.S. Pat. No. 5,672,176, the specification of which is incorporated herein by reference in its entirety. In mono-axial embodiments, the bone anchor and the receiving member may be coaxial or may be oriented at angle with respect to one another. In poly-axial embodiments, the bone anchor may biased to a particular angle or range of angles to provide a favored angle the bone anchor. Exemplary favored-angle bone screws are described in U.S. Patent Application Publication No. 2003/0055426 and U.S. Patent Application Publication No. 2002/0058942, the specifications of which are incorporated herein by reference in their entireties.

Therefore, in accordance with the present invention, and now referring to FIG. 3, there is provided a posterior dynamic spinal stabilization system for use in a human spine, comprising:
a) first and second bone anchors, each anchor having a recess for receiving a rod, and a set screw 101,
b) a first hollow cylinder having an open end, an intermediate annular portion and a closed end, the closed end defining an inner surface, the intermediate annular portion defining an outer annular surface and a first inner annular surface,
c) a first rod having an outer diameter, a first end having a first end surface, and a second end, the first end of the first rod being slidably received within the inner annular surface of the first hollow cylinder, and
d) a first elastomeric sleeve having a first end and a second end,
   wherein the first end of the first elastomeric sleeve is attached to the outer surface of the closed surface of the first hollow cylinder, and
   wherein the second end of the first elastomeric sleeve is attached to the outer surface of the second end of the inner rod,
   wherein the outer annular surface of the first hollow cylinder is received in the recess of the first bone anchor,
   wherein the second end of the first rod is received in the recess of the second bone anchor.

Generally, in using the present invention, two bone anchors such as polyaxial screws are inserted into adjacent pedicles within a functional spinal unit of a patient. The cylinder-bumper-rod assembly of the present invention is then inserted into the patient between the anchors. The first hollow cylinder is attached to the first bone anchor by laying the outer annular surface of the first hollow cylinder into the first bone anchor recess and tightening an appropriate set screw. Similarly, the second end of the first rod is attached to the second bone anchor by laying the second end into the second bone anchor recess and tightening the appropriate set screw. More preferably, this is achieved in a minimally invasive surgery.

In some embodiments, at least one end of the cylinder-bumper-rod assembly has a bullet nose for ease of insertion.

In some embodiments, the assemble may be implanted in accordance with the minimally invasive techniques and instruments disclosed in U.S. Pat. No. 7,179,261; and US Patent Publication Nos. US2005/0131421; US2005/0131422; US 2005/0215999; US2006/0149291; US2005/0154389; US2007/0233097; and US2005/0192589, the specifications of which are hereby incorporated by reference in their entireties.

Therefore, in accordance with the present invention, there is provided a method of implanting a posterior dynamic spinal stabilization system, comprising the steps of:
a) inserting two bone anchors into adjacent pedicles within a functional spinal unit of a patient, each bone anchor having a recess for receiving a rod,
b) providing a dynamic stabilization device comprising:
   i) a first hollow cylinder having an open end, an intermediate annular portion and a closed end, the closed end defining an inner surface, the intermediate annular portion defining an outer annular surface and a first inner annular surface,
   ii) a first rod having an outer diameter, a first end having a first end surface, and a second end, the first end of the first rod being slidably received within the inner annular surface of the first hollow cylinder, and
   iii) a first elastomeric sleeve having a first end and a second end,
      wherein the first end of the first elastomeric sleeve is attached to the outer surface of the closed surface of the first hollow cylinder, and
      wherein the second end of the first elastomeric sleeve is attached to the outer surface of the second end of the inner rod,
c) fastening the outer annular surface of the first hollow cylinder into the recess of the first bone anchor, and
d) fastening the second end of the first rod into the recess of the second bone anchor.

Preferably, the rods may be constructed of biocompatible metals such as titanium alloy, stainless steel and cobalt-chrome. Preferably, a very hard metal such as cobalt-chrome is selected so that the surface can be highly polished and provide good wear properties. In preferred embodiments, the surface finish Ra for the telescoping metallic surfaces should be no more than Ra 0.25 μm. In preferred embodiments, the surface finish Ra for the telescoping polymeric surfaces should be no more than Ra 0.50 μm.

Preferably, the articulating surfaces of the rod and hollow cylinder may be coated with materials such as diamond-like carbon, chromium nitride and titanium nitride. These coatings advantageously create a harder, more wear resistant surface.

Preferably, the rods may be constructed of a polymeric material such as carbon-reinforced PEEK to enable the rods to allow slight bending, which may prevent the components from binding.

Each component of the design may be made from biocompatible, implantable materials known in the art such as stainless steel, titanium, Nitinol, polyetheretherketone (PEEK) or alternative polyarylketones, carbon fiber reinforced polymers, and high performance elastomers such as silicones, dimethylsiloxanes, silicone-urethanes, polyether-urethanes, silicone-polyether-urethanes, polycarbonate urethanes, and silicone-polycarbonate-urethanes.

Preferably, the hollow cylinder components are titanium alloy (Ti-6Al-4V) or cobalt-chrome alloy (e.g. Co—Cr—Mo). If a cobalt-chrome alloy is selected, the alloy is preferably in a work-hardened condition so as to resist deformation upon securing to the bone anchor (e.g with a set screw). Preferably, the solid rod component is either titanium alloy or PEEK. More preferably, the hollow cylinder and solid rod components are selected such that articulation between the two components causes minimal wear, e.g. PEEK solid rod component with titanium alloy hollow cylinder component, or titanium alloy solid rod component with cobalt-chrome hollow cylinder component.

If a metal is chosen as a material of construction, then the metal is preferably selected from the group consisting of nitinol, titanium, titanium alloys (such as Ti-6Al-4V), cobalt-chrome alloys (such as CrCo or Cr—Co—Mo) and stainless steel.

If a polymer is chosen as a material of construction, then the polymer is preferably selected from the group consisting of polycarbonates, polyesters, (particularly aromatic esters such as polyalkylene terephthalates, polyamides; polyalkenes; poly(vinyl fluoride); PTFE; polyarylethyl ketone PAEK; and mixtures thereof.

In some embodiments, the tube and/or solid rod component is made from a composite comprising carbon fiber. Composites comprising carbon fiber are advantageous in that they typically have a strength and stiffness that is superior to neat polymer materials such as a polyarylethyl ketone PAEK. In some embodiments, the tube is made from a polymer composite such as a PEKK-carbon fiber composite.

Preferably, the composite comprising carbon fiber further comprises a polymer. Preferably, the polymer is a polyarylethyl ketone (PAEK). More preferably, the PAEK is selected from the group consisting of polyetherether ketone (PEEK), polyether ketone ketone (PEKK) and polyether ketone (PEK). In preferred embodiments, the PAEK is PEEK.

In some embodiments, the carbon fiber comprises between 1 vol % and 60 vol % (more preferably, between 10 vol % and 50 vol %) of the composite. In some embodiments, the polymer and carbon fibers are homogeneously mixed. In others, the material is a laminate. In some embodiments, the carbon fiber is present in a chopped state. Preferably, the chopped carbon fibers have a median length of between 1 mm and 12 mm, more preferably between 4.5 mm and 7.5 mm. In some embodiments, the carbon fiber is present as continuous strands.

In especially preferred embodiments, the composite comprises:

a) 40-99% (more preferably, 60-80 vol %) polyarylethyl ketone (PAEK), and
b) 1-60% (more preferably, 20-40 vol %) carbon fiber,
wherein the polyarylethyl ketone (PAEK) is selected from the group consisting of polyetherether ketone (PEEK), polyether ketone ketone (PEKK) and polyether ketone (PEK).

In some embodiments, the composite consists essentially of PAEK and carbon fiber. More preferably, the composite comprises 60-80 wt % PAEK and 20-40 wt % carbon fiber. Still more preferably the composite comprises 65-75 wt % PAEK and 25-35 wt % carbon fiber.

The elastomer bumper component is preferably made of a thermoplastic, biocompatible, high performance polycarbonate-urethane (PCU). The stiffness, or durometer of the PCU can be tailored to meet the specifications for the dynamic device. In preferred embodiments, the surface of the device components that will be attached to the elastomer bumper are treated prior to attaching the bumper using known surface treatment methods such as surface roughening (e.g. grit blasting), chemical functionalization (e.g. primers), and plasma treatments know in the art. Alternatively or in conjunction with using a surface treatment, an adhesive may be used to enhance bonding, e.g. using cyanoacrylates. In one preferred embodiment, the surfaces of the device components that will attached to the elastomer bumper will first be roughened using grit blasting, then chemically functionalized using primer, then the elastomer will be overmolded onto the device components.

We claim:

1. A dynamic stabilization device comprising:
a) a first hollow cylinder having an open end, an intermediate annular portion and a closed end, the closed end defining an inner surface, the intermediate annular portion defining an outer annular surface and a first inner annular surface,
b) a first rod having an outer diameter, a first end having a first end surface, and a second end, the first end of the first rod being slidably received within the inner annular surface of the first hollow cylinder, and
c) a first elastomeric sleeve having a first end and a second end,
wherein the first end of the first elastomeric sleeve is attached to the outer surface of the closed surface of the first hollow cylinder, and
wherein the second end of the first elastomeric sleeve is attached to the outer surface of the second end of the first rod.

2. The device of claim 1 further comprising a mechanical means to fasten the outer elastomer jacket to the inner rod and outer hollow cylinder.

3. The device of claim 2 wherein the mechanical means is selected from the group consisting of a groove, a ridge, boss and a set screw.

4. The device of claim 1 further comprising a bullet nose on the first end surface of the inner rod, and the inner surface of the hollow cylinder has a corresponding cup shape.

5. The device of claim 1 wherein the inner rod has an intermediate section having a transition radius region.

6. The device of claim 1 wherein the inner rod and outer hollow cylinder have corresponding non-circular cross-sections.

7. The device of claim 6 wherein the non-circular cross-sections are selected from the group consisting of an ellipse, a square, a polygon, and T-slotted.

8. The device of claim 1 wherein the inner rod and outer hollow cylinder are curved to provide lordosis.

9. The device of claim 1 wherein the outer diameter of the inner rod has a pin extending therefrom and the outer hollow cylinder has a corresponding groove, wherein the pin is received in the groove.

10. The device of claim 1 wherein the inner rod and outer hollow cylinder have slotted features at their ends to allow the rod to mate with bone anchors that contain posts.

11. The device of claim 1 further comprising a deformable thin metallic sleeve provided around the elastomeric sleeve.

12. The device of claim 1 further comprising an elastomeric ring assembled to the outside of the inner rod at its first end.

13. The device of claim 1 further comprising a hole adapted to provide escape of telescopic air movement.

14. The device of claim 13 wherein the set screw has a washer or rotating saddle shape feature at an inferior surface that would allow axial load to be applied to the elastomeric jacket without transferring torque.

15. The device of claim 13 wherein the set screw has a boss at the center, wherein the boss is adapted to penetrate the elastomeric sleeve and minimize torque thereto.

16. The device of claim 1 wherein at least one of the outer hollow cylinder or inner rod comprises an attachment feature for attachment to a minimally invasive surgery (MIS) instrument.

17. A dynamic stabilization device comprising:
a) a first hollow cylinder having an open end, an intermediate annular portion and a closed end, the closed end defining an inner surface, the intermediate annular portion defining an outer annular surface and a first inner annular surface,
b) a first rod having an outer diameter, a first end having a first end surface, and a second end, the first end of the first rod being slidably received within the inner annular surface of the first hollow cylinder, and
c) a second hollow cylinder having an open end, an intermediate annular portion and a closed end, the closed end defining an inner surface, the intermediate annular portion defining an outer annular surface and a second inner annular surface,
d) a first elastomeric sleeve having a first end and a second end,
wherein the first end of the first elastomeric sleeve is attached to the closed end of the first hollow cylinder, and
wherein the second end of the first elastomeric sleeve is attached to the closed end of the first hollow cylinder.

18. A posterior dynamic spinal stabilization system for use in a human spine, comprising:
a) first and second bone anchors, each anchor having a recess for receiving a rod,
b) a first hollow cylinder having an open end, an intermediate annular portion and a closed end, the closed end defining an inner surface, the intermediate annular portion defining an outer annular surface and a first inner annular surface,
c) a first rod having an outer diameter, a first end having a first end surface, and a second end, the first end of the first rod being slidably received within the inner annular surface of the first hollow cylinder, and
d) a first elastomeric sleeve having a first end and a second end,
wherein the first end of the first elastomeric sleeve is attached to the outer surface of the closed surface of the first hollow cylinder, and
wherein the second end of the first elastomeric sleeve is attached to the outer surface of the second end of the inner rod,
wherein the outer annular surface of the first hollow cylinder is received in the recess of the first bone anchor,
wherein the second end of the first rod is received in the recess of the second bone anchor.

19. A method of implanting a posterior dynamic spinal stabilization system, comprising the steps of:
a) inserting two bone anchors into adjacent pedicles within a functional spinal unit of a patient, each bone anchor having a recess for receiving a rod,
b) providing a polyaxial dynamic stabilization device comprising:
i) a first hollow cylinder having an open end, an intermediate annular portion and a closed end, the closed end defining an inner surface, the intermediate annular portion defining an outer annular surface and a first inner annular surface,
ii) a first rod having an outer diameter, a first end having a first end surface, and a second end, the first end of the first rod being slidably received within the inner annular surface of the first hollow cylinder, and
iii) a first elastomeric sleeve having a first end and a second end,
wherein the first end of the first elastomeric sleeve is attached to the outer surface of the closed surface of the first hollow cylinder, and
wherein the second end of the first elastomeric sleeve is attached to the outer surface of the second end of the inner rod,
c) fastening the outer annular surface of the first hollow cylinder into the recess of the first bone anchor, and
fastening the second end of the first rod into the recess of the second bone anchor.

20. A dynamic stabilization device comprising:
a) a first rod having an outer diameter, an inner end having an inner end surface, and an outer end, the inner end of the first rod being slidably received within the first inner annular surface of the dual hollow cylinder, and
b) a second rod having an outer diameter, an inner end having an inner end surface, and an outer end, the inner end of the second rod being slidably received within the second inner annular surface of the dual hollow cylinder, and
c) a dual hollow cylinder having an first and second open ends defining first and second annular portions, an intermediate solid portion, the first and second annular portions defining first and second outer annular surfaces and first and second inner annular surfaces,
d) a first elastomeric sleeve having a first end and a second end,
wherein the first end of the first elastomeric sleeve is attached to the outer end of the first rod, and
wherein the second end of the first elastomeric sleeve is attached to the outer end of the second rod.

21. A Multi-level Dynamic Stabilization device comprising:
a) a first hollow cylinder having an open end, an intermediate annular portion and a closed end, the closed end defining an inner surface, the intermediate annular portion defining an outer annular surface and a first inner annular surface,
b) a first rod having an outer diameter, a first end having a first end surface, and a second end having a second end surface.
c) a third component with a first rod end, and second hollow cylinder with an open end defining an inner surface, the annular portion defining an outer annular surface and an inner annular surface, the first rod end containing a first end forming a first outer diameter to be slidably received within the inner annular surface of the first hollow cylinder and the second hollow end slidably mated to the first rod.
d) a first elastomeric sleeve having a first end and a second end, wherein the first end of the first elastomeric sleeve is attached to the closed end of the first hollow cylinder and the second end of the first elastomeric sleeve attached to the second end surface of the first rod.
